(19) United States
(12) Patent Application Publication
(10) Pub. No.: US 2009/0137960 A1
(43) Pub. Date: May 28, 2009

(54) Access Device For Medical Purposes, Method For The Production Thereof and an Access System Including Such an Access Device

(76) Inventors: Roger Johansson, Knivsta (SE); Dick Persson, Los (SE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/087,740

(22) PCT Filed: Nov. 29, 2006
(86) PCT No.: PCT/SE2006/001362

§ 371 Date: Oct. 6, 2008

(30) Foreign Application Priority Data
Jan. 17, 2006 (SE) 060081-4

(51) Int. Cl. A61M 5/32; B23P 11/00

(52) U.S. Cl. 604/175; 604/174; 29/428

(57) ABSTRACT

An access device (2) for external connection of an external circuit or a fluid source to a patient for medical purposes, including mouths (7) of access channels (8) with connections that are connectable to the patient and being surrounded by a connecting surface (6), and a guide with holding elements (5) which is open in its both ends for the co-operation with holding means (17) on a coupling device (3,4). Portions (30) of the access channels (8), which are most adjacent to each other are formed with curves that are directed to each other so that the course of a transition channel (9), which is arranged to be formed by the access device and a connected coupling device of the kind which is formed in order to directly interconnect said two mouths, has an inner curved portion in the area of the connecting surface. The invention also concerns a method for producing an access device, a coupling device and an access system.

### FIELD OF THE INVENTION

The present invention concerns an access device according to the preamble of claim 1. It also concerns a method for production thereof, a coupling device and an access system including such an access device.

### BACKGROUND OF THE INVENTION

WO 99/20338 concerns an access device having connecting surfaces surrounding connecting openings, which are intended to co-operate with connecting surfaces surrounding channel openings on a coupling device communicating with an external circuit. The connecting surfaces of the access device as well as of the coupling device are covered in a non connected position and protected by a kind of lid or the like, which during the connection of a coupling device of any type are arranged to be moved away from the respected connecting surfaces.

The purpose of protecting the connecting surfaces is to avoid contamination thereof and thereby avoid or at least radically reduce risks of infection for a patient utilizing the coupling.

The device which is known from the cited document function well but it has been brought to notice a desire to achieve better flow through the device. Further, the previously known device is relatively expensive to produce.

### AIM AND MOST IMPORTANT FEATURES

It is an aim of the present invention to avoid the said problems of the background art and to provide a development of the previously known device which with maintained high hygienic standard further is more uncomplicated to produce, simple to handle and can be produced to a reduced cost.

These aims are achieved in a device as above through the features of the characterized portion of claim 1.

Hereby is achieved enhanced flow geometry of the device and the access device is equipped with a coupling device in a form of a lid, which directly interconnects the mouths.

It is thus allowed that the access channels, at least in the area of the mouths, can be rectilinear and as most preferred example have a rectilinear extension and form an angle close to 90° with the connecting surface.

By means of the gentle inner curve, turbulence in the flow is prevented or at least reduced, which besides reducing flow resistance also reduces the risk of the activation of coagulation. The access channels can by means of the inventive design of the access device thus be produced with preferred short length and relatively large cross section, which aspects both contribute to reduce contact surface between blood and materials that are foreign for the body (concerns the use as a blood vessel valve).

It is preferred that the inner curve is entirely formed in the access device, which gives the best flow characteristics, but it is not excluded that a portion of the inner curve is defined by a coupling device intended to be applied thereon. This latter solution allows simpler design of the access device but more complicated coupling devices.

It is preferred that the access channels have essentially rectilinear extension which gives low flow resistance and is easy to produce.

If a tangent of the curves, where adjoining to a portion of the channel, have a direction essentially corresponding to the direction of a generatrix thereof, a gentle transition between these portions is achieved and an advantageous flow. Hereby is understood that an angular deviation is tolerable.

When a tangent of each one of said curves, where joining to the connecting surface, has a direction essentially corresponding to the direction thereof, the corresponding gentle transition is obtained and favourable flow. Hereby is to be understood that an angular deviation is tolerable.

When said inner curve in its entirety is formed in the access device, the shortest flow path is obtained for fluid flowing in the transition channel. The total height of an access device with mounted short-circuiting unit will also be lower, which is an advantage in case of an implant.

It is preferred that the radius of curvature for each inner curve is relatively large. Only as an example thereof it is proposed that the radius of curvature is about 15%-60% of the closest mutual distance between the portions of the access channels adjacent to said inner curves for at least a part of the curve. This results in relatively large radiuses of curvature and good flow properties.

By the access device being designed with a trough-shaped portion having a bottom, an edge portion and a trough opening, whereby each connection forms a pipe portion which protrudes from the bottom towards the opening, a high degree of manufacture friendliness is achieved and thereby advantageous economy. The design will also be material saving. The device can further be made more compact, since the connections to grafts or the like are positioned inside the trough-shaped portion. Hereby it is preferred that a free end of each pipe portion is positioned such that it does not reach a plane through the trough opening, which gives a protected variant of the connections and connected grafts and hoses.

In the use as a vessel valve for haemodialysis and in a plurality of other applications, i.e. the application of types of artificial organs other than artificial kidneys, in gas exchange such as oxidizing etc there is a need of two connections of the access device to blood vessels of the patient, wherein the present invention is advantageously used.

The design of the access device allows thus an effective channeling, which can reduce flow resistance in the device further compared to devices according to a background art. All in all, the device according to the invention is particularly preferred as a vessel valve.

### BRIEF DESCRIPTION OF DRAWINGS

The invention will now be described in greater detail by way of embodiments and with reference to the annexed drawings, wherein:

FIG. 1 shows parts of an access system including coupling devices according to the invention,

FIG. 2 shows an access device for the connection of a coupling device according to the invention, and

FIG. 3 shows, partly in section, an access device according to the invention with a connected coupling device.

### DESCRIPTION OF EMBODIMENTS

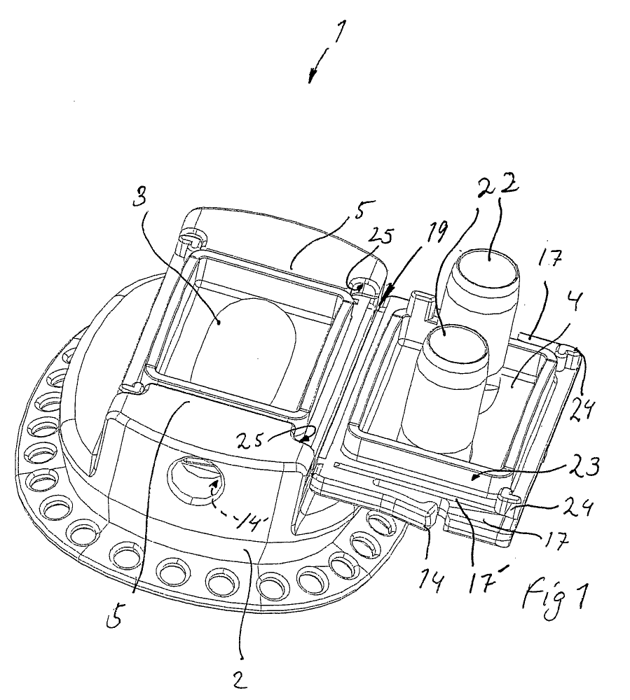

FIG. 1 shows an access system with a so called application device, different connections to external circuits such as blood renal systems etc. are eliminated for the sake of clarity. On an access device 2 is held a coupling device 3 of a first embodiment as a short-circuiting unit. Sideward of this short-circuiting unit 3 is shown a coupling device 4 according to a second embodiment which is as a coupling unit having connections 22 to an external circuit such as an artificial kidney.

For better understanding of the general purpose of an access device according to the invention reference is made to the applicant's own WO 99/20338, wherein also the principle of the function of an application device is described.

The access device 2 has first and second holding elements for the co-operation with holding means 17 which are arranged transversally of the respective coupling devices 3 and 4, and which extends over the width of the side of the respective coupling device 3 and 4. The holding elements 5 form a guide for coupling devices that is open in both ends which is shown in more detail in said WO document. On the holding means 17, there are arranged strips 17′ (one shown in the figure) for vertical (as seen in the figure) positioning of the coupling device against the holding surface of the holding elements 5.

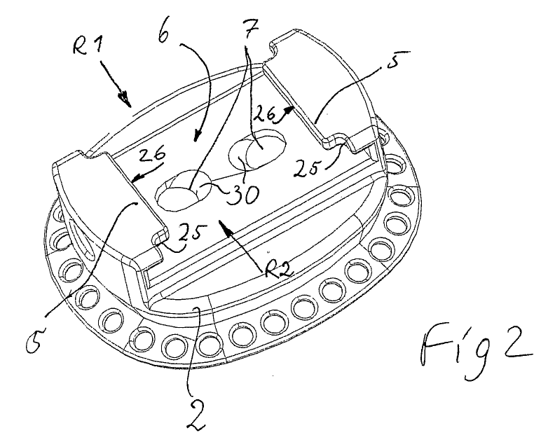

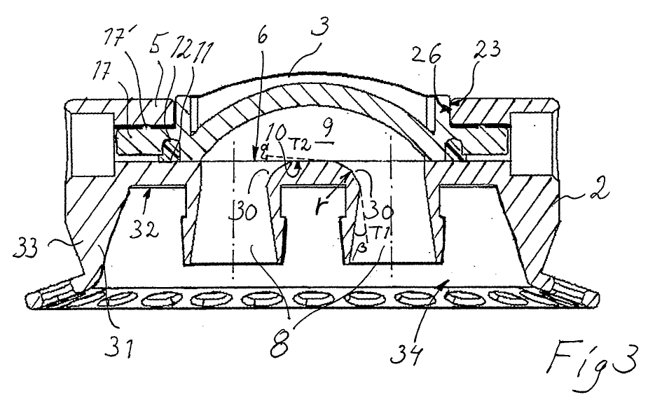

At an angle of a coupling surface (10 in FIGS. 3, 4 and 5), preferably at about right angle, extends on each side a positioning surface 23, which is arranged to co-operate with a positioning edge (26 in FIGS. 2 and 3) on the holding element 5 in order to achieve an adequate positioning in horizontal direction transversal to a direction of inserting the coupling device. For positioning in an insertion direction, there are arranged firstly snap lock arms 14 on the coupling device 4, that co-operate with holding edges (indicated at 14′) on the access device 2. Secondly, the coupling device 4 has stop lugs 24, which co-operate with stop recesses 25 on the access device, at the ends of each holding element. This way, good precision during application is ensured, which increases the precision of the channel formed by the access device and the coupling device, so that thereby protruding edges and recesses that negatively affect the flow because of erroneous positioning are avoided.

FIG. 2 shows the access device 2 in greater detail, whereby application directions for coupling devices are indicated with arrows R1 and R2, respectively. With 6 is indicated a connecting surface and with 7 two mouths to two access channels for the connection to a patient.

FIG. 3 shows in a section an access device 2 with positioned coupling device 3 in the form of a short-circuiting unit of the kind including a transition channel portion for forming a transition channel 9 together with the access device. In the figure is shown that the holding elements 5 co-operate with the holding means 17 and in particular the strip 17′ for adequate positioning in a vertical direction as seen in the figure. With 6 is indicated the connecting surface of the access device and with 10 a coupling surface of the coupling device 3.

Outward of the short-circuiting channel 9 is positioned a surrounding groove 12 for receiving a strip-shaped means comprising a flexible portion 11, which is arranged with one part to protrude past, or outside the plane of said coupling surface 10. The flexible portion 11 comprises a sealing lip (not shown here), which has a more limited, in the protrusion direction narrowing extension. The sealing lip works well in order to take up possible tolerance deviations of the involved parts. It is, however, not part of this invention.

Portions of the access channels, which are most adjacent to each other are indicated with 30 and are formed with curves directed to each other such that the extension of the transition channel 9, which is formed by the access device 2 and the coupling device 3 has an inner curved portion in the area of the connecting surface.

The pipe-shaped portions, which form the access channels 8 have axes with essentially rectilinear extension, and the channels are here shown widening in the direction from the connecting surface. The axes of the pipe-shaped portions form preferable 90° or almost 90° angle to the adjacent portions of the connecting surface for achieving manufacture friendliness and good flow properties when applying different types of coupling devices.

The manufacture friendliness is accentuated when the axes of the pipe-shaped portions are essentially parallel to each other, since this results in simpler forming of the channels in the access device for example when the channels are produced by milling operations.

By a tangent T1 of each one of said curves where adjoining a portion of the channel has a direction which closely corresponds to the direction of a generatrix of the channel in this region, a gentle transition between these portions is obtained and a corresponding favourable flow. The corresponding is true if a tangent T2 of each one of said curves where adjoining to the connecting surface has a direction essentially corresponding to the direction thereof. Certain angular deviation α and β, respectively in FIG. 3 is tolerable, but it is preferred that there is a soft transition between curved portions and straight portions. This means that the radius of curvature r can be greater than 50% of the closest distance between the mouths of the channels, and it is within the scope of the invention that the radius of curvature can be at least 60% of this distance and even considerable larger even if 50% of the distance would be preferred. Within the invention is also that the radius of curvature varies over the course of the curve such that for example a greater radius of curvature is present centrally of the curve and smaller radius of curvature is present at least at one “end” of the curve, that is closest to the connecting surface of the straight extension of the channel.

In the shown embodiment, said inner curve is entirely formed in the access device. It is also within the scope of the invention that the access device is formed such that a portion of the inner curve which is formed between two access channels is present inside the coupling device that is the latter forms a complete enclosed pipe portion. This gives the possibility of a more continuous extension of the formed channel but builds in height, which normally is not preferred. Further, the production of the coupling devices shaped this way will be more complicated.

The radius of curvature r for the inner curve is in the shown example held large in order to enhance flow properties through the device.

The access device is shaped with a trough-shaped portion 31 having a bottom 32, an edge portion 33 and a trough opening 34, wherein each connection forms a pipe portion been defined of one of said access channels 8, which extends from the bottom 32 towards the trough opening 34, which in turn is directed essentially opposite the connecting surface. A free end of each pipe portion is positioned such that it does not reach a plane through the trough opening. The pipe portions have contact means in the form of enlargements at their outer parts in order to better co-operate with the grafts, hoses etc.

The connecting surface 6 is plane in the shown example and this is preferred. Embodiments having a curved surface are, however, possible.

The shown access device is implantable and has means in the form of a surrounding ingrowth collar for supporting the ingrowth of human tissue, which extends outwardly in the outside of the opening of the trough-shaped portion.

In the areas of each holding elements 5, the access device is provided with a hole, which besides with the wall 14′ co-operate with a snap lock arms 14 on the coupling device 3 and 4 allows access to the connecting surface for cleaning purposes.

A method for production of an access device 2 according to the invention is distinguished by portions of the access channels which are most adjacent to each other are shaped having curves directed to each other such that the extension of a transition channel, which is arranged to be formed by the access device and a coupling device of the kind which is designed to directly connect said mouths, is giving an inner curved portion in the area of the connecting surface. In particular, the access device is provided with said curves 30 in the access channel by a milling operation, more particularly plunge-cutting milling machining, wherein for example a program controlled milling machine produces the gentle curve.

A blank which is given said inner curved portion 30 can previously have been produced in different ways such as in machining operation including cutting such as milling or turning. It is also possible to precision cast, for example die-cast the blank, wherein in that case the other surfaces besides the “curved surfaces” of the access device are finished directly.

The invention can be modified within the scope of the following claims.

Access devices according to the invention can be of different kinds besides the one mentioned above and co-operate with different kinds of coupling devices. Thus, a coupling device for connecting the external circuit and be used for extracorporeal blood treatment such as for example haemodialysis. A coupling device for connecting to a fluid circuit source can be used for medication, supply of nutrient etc. Further, the access device can co-operate with coupling devices including sensor elements, for example for temperature control, which can be used on the one hand for monitoring the function of the access device, on the other hand certain conditions of the patient. Other parameters in the blood can be monitored, for example concentration of different substances. This can be indicated with the aid of different kinds of signal systems.

A coupling device 3 in the form of a short-circuiting unit has its transition channel portion (its part of transition channel 9) shaped for adaptation to the access device in order as much as possible to form a transition channel extending as evenly as possibly. For this purpose the dimension parameters such as radiuses of curvature along the transition channel portion, transversally of the transition channel portion etc. are made for obtaining a similar cross sectional area and cross sectional geometry over the length extension of the transition channel and as much as possible avoid abrupt flow path alterations.

The invention can be varied in the scope of the following claims and is not limited to shown embodiment. Thus, the invention can be applied in a device having one single connection and then as an implant as well as a hose connection used outside the human body.

Also other and otherwise shaped components can be used to be connected to the access device, for example a membrane unit which can have a design essentially as the coupling device 3, but where a penetrable membrane delimits the channel 9 from the surroundings. As an alternative, a separate membrane unit can be connected to the coupling device through hose (hoses).

Forming of details can deviate from the shown embodiment. Thus the guide can be shaped otherwise. The guide can be curved, for example circular and the connecting surfaces can be positioned on a curved surface as seen transversally to the connection direction.

The positioning of and equipment with snap means such as snap lock arms etc. can deviate from what is shown. Also the elements for releasing a snap locked coupling device or a protective part in the form of simpler lid can be varied with respect to shape as well as positioning.

The device can be used as an implant or separate coupling device having one or two outgoing connections as well as openings and conduit channels in the respective part.

Materials that are used are bio-compatible to the extent that is required for the application. By the inventive construction of an access device according to the invention it is well suited for manufactured in titanium or corresponding metallic materials or alloys. It is also possible to produce the access device from synthetic materials such as plastics and also from ceramic materials. Possibly the surfaces of the access device can be treated with blood compatible or/and bio-compatible coverings. The parts included in an access system according to the invention can be comprised of disposable articles which are producible at a low price. This goes particularly for different kinds of coupling devices.

1. Access device (2) for external connection of an external circuit or a fluid source to a patient for medical purposes, including mouths (7) of access channels (8) with connections that are connectable to the patient and being surrounded by a connecting surface (6), and a guide with holding elements (5) which is open in its both ends for the co-operation with holding means (17) on a coupling device (3,4), characterized in
that portions (30) of the access channels (8), which are most adjacent to each other are formed with curves that are directed to each other so that the course of a transition channel (9), which is arranged to be formed by the access device and a connected coupling device of the kind which is formed in order to directly interconnect said two mouths, has an inner curved portion in the area of the connecting surface.

2. Device according to claim 1, characterized in that the access channels (8) are widening in the direction from the connecting surface.

3. Device according to claim 2, characterized in that the axes of the access channels (8) have essentially rectilinear extension and/or are essentially parallel.

4. Device according to any of the previous claims, characterized in that the axes of the access channels (8) form essentially right angle to the connecting surface.

5. Device according to any of the previous claims, characterized in that a tangent (T1) of each one of said curves, where adjoining to a portion of an access channel, has a direction essentially corresponding to the direction of a generatrix thereof in order to obtain a gentle transition between these portions.

6. Device according to any of the previous claims, characterized in that a tangent (T2) of each one of said curves, where adjoining to the connecting surfaces (6) has a direction essentially corresponding to the direction thereof for obtaining a gentle transition there between.

7. Device according to any of the previous claims, characterized in that said inner curve is entirely formed in the access device (2).

8. Device according to any of the previous claims, characterized in that said inner curve is partially formed in the access device (2), which is formed for co-operation with a coupling device, which in turn has an inner curve.

9. Device according to any of the previous claims, characterized in that a radius of curvature for at least a part of each inner curve is about 15%-60% of the closest mutual distance between portions of the access channels (8) adjacent to said inner curves.

10. Device according to any of the previous claims, characterized in that the access device (2) is formed with a trough-shaped portion (31) having a bottom (32), an edge portion (33) and a trough opening (34), wherein each connection forms a pipe portion which extends from the bottom (32) against the trough opening (34).

11. Device according to claim 10, characterized in that the free end of each pipe portion is positioned such that it does not reach a plane through the trough opening (34).

12. Device according to claim 10 or 11, characterized in that each pipe portion is provided with contact means for connection with a graft.

13. Device according to any of the claims 10-12, characterized in that each connecting surface (6) is directed essentially opposite to the trough opening (34).

14. Device according to any of the previous claims, characterized in that each connecting surface (6) is plane.

15. Device according to any of the previous claims, characterized in that the access device (2) is implantable and comprises means for supporting the ingrowth of human tissue.

16. Device according to claim 15, characterized in that the means for supporting ingrowth is an ingrowth collar.

17. Device according to any of the claims 10-16, characterized in that the ingrowth collar extends outwardly from the outside of the trough-shaped portion.

18. Device according to claim 17, characterized in that the ingrowth collar extends outwardly from the opening of the trough-shaped portion.

19. Device according to claim 17 or 18, characterized in that the ingrowth collar extends surrounding.

20. Device according to any of the previous claims, characterized in that in the areas of holding elements (5) it comprises a hole in order to allow access to the connecting surface for cleaning purposes.

21. Coupling device for connection to an access device (2) according to any of the claims 1-20, characterized in that it comprises a transition channel portion shaped with transversal and longitudinal radiuses of curvature which are dimension such that the course of a transition channel (9) which is to be formed by the access device and a coupling device has evenly extending transversal sectional area and transversal sectional geometry along the longitudinal extension.

22. Access system including at least one coupling device and one access device according to any of the claims 1-20.

23. Method for producing an access device (2) according to any of the previous claims, characterized in
that portions of the access channels which are most adjacent to each other are formed having curves directed to each other such that the course of a transition channel which is arranged to be formed by the access device and a coupling device of the kind which is shaped in order to directly interconnect said two mouths is provided with an inner curved portions in the area of the connections surface.

24. Method according to claim 23, characterized in that said curves in the access channels are brought into the access device by means of a milling operation, in particular plunge cutting milling.
